(19) United States
(12) Patent Application Publication
(10) Pub. No.: US 2015/0064117 A1
(43) Pub. Date: Mar. 5, 2015

(54) NOVEL POLYMORPHS OF REBAUDIOSIDE C AND METHODS FOR MAKING AND USING THE SAME

(71) Applicant: Redpoint Bio Corporation, Ewing, NJ (US)

(72) Inventors: F. Raymond Salemme, Yardley, PA (US); Robert A. Daines, Lansdale, PA (US)

(73) Assignee: Redpoint Bio Corporation, Ewing, NJ (US)

(21) Appl. No.: 14/530,056

(22) Filed: Oct. 31, 2014

Related U.S. Application Data
(63) Continuation of application No. 13/497,619, filed on May 17, 2012.
(60) Provisional application No. 61/244,803, filed on Sep. 22, 2009.

(51) Int. Cl. A23L 1/236; A61K 47/26; A61K 8/60; A61Q 11/00; A23L 1/30; C07H 15/24

(52) U.S. Cl. CPC A23L 1/2366; A23L 1/3002; C07H 15/24; A61K 8/60; A61Q 11/00; A61K 47/26; A23V 2002/00; A61K 2800/10
USPC 424/49; 426/2; 426/658; 536/18.1; 514/777

(57) ABSTRACT

Embodiments of this invention encompass a method for producing and purifying rebaudioside C. In particular, this invention relates to a method for purifying rebaudioside C compositions to obtain a substantially pure rebaudioside C product using one or more crystallization steps. Resulting polymorphic forms of rebaudioside C, substantially pure rebaudioside C compositions and their uses are disclosed.

### INTRODUCTION

This application is a continuation of U.S. Ser. No. 13/497,619 filed May 17, 2012, which is a 371 application of PCT/US2010/049763 filed Sep. 22, 2010, which claims benefit of priority to U.S. Provisional Application Ser. No. 61/244,803 filed Sep. 22, 2009, the contents of which are incorporated herein by reference in their entireties.

### BACKGROUND OF THE INVENTION

The sweet diterpene glycosides of Stevia have been characterized, and eight sweet glycosides of steviol have been identified. These glycosides accumulate in Stevia leaves where they may attain from 10 to 20% of the leaf weight. On a dry weight basis, a typical profile for the four major glycosides found in the leaves of Stevia includes 0.3% dulcoside, 0.6% rebaudioside C, 3.8% rebaudioside A and 9.1% stevioside. Other glycosides identified within Stevia include rebaudiosides B, D, and E, and dulcosides A and B. Out of the four major diterpene glycoside sweeteners present in Stevia leaves only two (stevioside and rebaudioside A) have physical and sensory properties that are well characterized. Stevioside is known to be 110 to 270 times sweeter than sucrose, rebaudioside A 150 to 320 times sweeter than sucrose, rebaudioside C 40 to times sweeter than sucrose, and dulcoside A 30 times sweeter than sucrose.

Of the diterpene glycosides found in Stevia extracts, rebaudioside A is known to have the least aftertaste. This aftertaste is described by many as bitter and licorice-like, which is present in all current Stevia extracts.

Rebaudioside A has been tested in mixtures with other sweeteners, such as fructose, glucose and sucrose, at intensities equivalent to 3% (w/v-%), 5% (w/v-%) and 7% (w/v-%) sucrose to determine the presence and degree of synergism in these mixtures (Schiffmann et al., Brain Research Bulletin 38:105-120 (1995)). According to the results, rebaudioside A appears to have an additive effect in mixtures with fructose and glucose, but a synergistic effect in mixtures with sucrose at sweetness intensities equivalent to 3% (w/v-%) sucrose. At sweetness intensities equivalent to 5% (w/v-%), rebaudioside A had an additive effect in mixtures with fructose, glucose and sucrose. At sweetness intensities equivalent to 7% (w/v-%) sucrose, rebaudioside A had an additive effect with a mixture with sucrose, but a suppressive effect with mixtures with glucose and fructose. In fact, no sweetener combinations were synergistic at sweetness intensities equivalent to the 7% (w/v-%) sucrose level.

U.S. Pat. No. 4,612,942 mentions that diterpene glycosides can modify or enhance flavor characteristics, such as sweet, when the amount of diterpene glycoside added is less than the sweetness threshold level of the diterpene glycoside in the orally consumable composition.

Previously reported efforts to produce and purify rebaudioside C require numerous reaction steps or iterative purification steps. A need exists for providing a simple, efficient, and economical method for producing rebaudioside C using fewer purification steps. A need also exists for a rebaudioside C crystalline form that is fairly soluble, is stable, and can be better handled and blended.

### SUMMARY OF THE INVENTION

The present invention relates to substantially pure polymorphic forms of rebaudioside C, methods for purifying rebaudioside C, methods for making polymorphic forms of rebaudioside C, and to the use of polymorphic forms of rebaudioside C for enhancing the sweet taste of carbohydrate sweeteners, such as sucrose and fructose.

One aspect of the present invention is to provide different polymorphic forms of rebaudioside C, and especially rebaudioside C crystalline Form I (as shown in FIG. 2). Methods for preparing different polymorphic forms of rebaudioside C are also provided.

One aspect of the present invention provides a method for purifying rebaudioside C which comprises one or more crystallization steps. Preferably, this purification method comprises combining either a Stevia extract solid or a crude rebaudioside C solid with a crystallization solution comprising an organic solvent to form a crude rebaudioside C solution and crystallizing from the crude rebaudioside C solution, a substantially pure rebaudioside C solid with a purity of at least about 90% by dry weight. Preferably, the crystallization solution comprises methanol, ethanol, isopropanol, or mixtures thereof. The crystallization solution can comprise water, such as in an amount from about 5% to about 25% by weight. In the crystallization step of the purification method, rebaudioside C polymorph crystals can be used as seeds.

One aspect of the present invention is to provide a method of enhancing a sweet taste of a carbohydrate sweetener. This method comprises administering to a subject the carbohydrate sweetener and an effective amount of one or more rebaudioside C polymorphs, especially rebaudioside C crystalline Form I, wherein the effective amount provides a sweet taste enhancing effect without exhibiting any off-taste. Preferably, the carbohydrate sweetener is sucrose, fructose, or glucose. In one embodiment, the carbohydrate sweetener and one or more rebaudioside C polymorphs are administered in a consumable. The consumable includes, but is not limited to, a food product, a dietary supplement, a nutraceutical, a pharmaceutical composition, a dental hygienic composition or a cosmetic product. In one embodiment, one or more rebaudioside C polymorphs are present in the consumable at a concentration of from about 150 μM to about 600 μM. In one embodiment, one or more rebaudioside C polymorphs are present in the consumable at a concentration of from about 150 μM to about 350 μM. In one embodiment, one or more rebaudioside C polymorphs are present in the consumable at a concentration of from about 350 μM to about 600 μM. In one embodiment, one or more rebaudioside C polymorphs are present in the consumable at a concentration of from about 250 μM to about 350 μM, and preferably about 250 μM or about 300 μM. In one embodiment, the sweetness intensity of the consumable is equivalent to about 5-12% (w/v-%) sucrose solution. In one embodiment, the sweetness intensity of the consumable is equivalent to about 5-7% (w/v-%) sucrose solution. In another embodiment, the sweetness intensity of the consumable is equivalent to about 8-12% (w/v-%) sucrose solution. In one embodiment, the sweetness intensity of the consumable is equivalent to about 5% (w/v-%), about 6% (w/v-%), about 7% (w/v-%), or about 8% (w/v-%) sucrose solution. In one embodiment, the sweetness intensity of the consumable is equivalent to about 9% (w/v-%), about 10% (w/v-%), about 11% (w/v-%), or about 12% (w/v-%) sucrose solution.

One aspect of the present invention is to provide a consumable, comprising a carbohydrate sweetener and one or more rebaudioside C polymorphs, especially rebaudioside C crystalline Form I, in an amount effective to enhance the sweet taste of the carbohydrate sweetener without exhibiting an off-taste. In one embodiment, the consumable of the present invention contains from about 150 μM to about 600 μM of one or more rebaudioside C polymorphs. In one embodiment, the consumable of the present invention contains from about 150 μM to about 350 μM, from about 250 μM to about 350 μM, and preferably about 250 μM or about 300 μM of one or more rebaudioside C polymorphs. In one embodiment, the consumable of the present invention contains from about 350 μM to about 600 μM of one or more rebaudioside C polymorphs. In one embodiment, the consumable has a sweetness intensity equivalent to about 5-12% (w/v-%) sucrose solution. In one embodiment, the consumable has a sweetness intensity equivalent to about 5-7% (w/v-%) sucrose solution. In another embodiment, the consumable has a sweetness intensity equivalent to about 8-120 (w/v-%) sucrose solution. In one embodiment, the sweetness intensity of the consumable of the present invention is equivalent to about 5% (w/v-%), about 6% (w/v-%), about 7% (w/v-%), about 8% (w/v-%), about 9% (w/v-%), about 10% (w/v-%), about 11% (w/v-%), or about 12% (w/v-%) sucrose solution.

Another aspect of the present invention is to provide a method of decreasing the amount of a carbohydrate sweetener in a consumable, comprising adding one or more rebaudioside C polymorphs, especially rebaudioside C crystalline Form I, to the consumable and thereby reducing the amount of the carbohydrate sweetener needed to exhibit a given level of sweetness.

In one aspect, the present invention provides a tabletop sweetener composition, comprising (i) at least one carbohydrate sweetener, (ii) one or more rebaudioside C polymorphs, especially rebaudioside C crystalline Form I; and (iii) optionally a bulking agent. Desirably, the one or more rebaudioside C polymorphs are each present in an amount effective to synergistically enhance the sweetness of the carbohydrate sweetener.

In another aspect, the present invention provides a tabletop sweetener composition consisting essentially of (i) at least one carbohydrate sweetener, (ii) one or more rebaudioside C polymorphs, especially rebaudioside C crystalline Form I; and (iii) optionally a bulking agent, wherein the one or more rebaudioside C polymorphs are each present in an amount effective to synergistically enhance the sweetness of the carbohydrate sweetener.

In one aspect, the present invention provides a method of making a tabletop sweetener composition, comprising including (i) at least one carbohydrate sweetener, (ii) one or more rebaudioside C polymorphs, especially rebaudioside C crystalline Form I, and (iii) optionally a bulking agent. In one embodiment, the one or more rebaudioside C polymorphs are included in an amount effective to synergistically enhance the sweetness of the carbohydrate sweetener. In a particular embodiment, the one or more rebaudioside C polymorphs are each independently at a concentration of from about 150 μM to about 600 μM. In one embodiment, one or more rebaudioside C polymorphs are present in the tabletop sweetener composition at a concentration of from about 150 μM to about 350 μM. In one embodiment, one or more rebaudioside C polymorphs are present in the tabletop sweetener composition at a concentration of from about 350 μM to about 600 μM. In one embodiment, one or more rebaudioside C polymorphs are present in the tabletop sweetener composition at a concentration of from about 250 μM to about 350 μM, and preferably about 250 μM or about 300 μM.

Another aspect of the present invention is to provide a method of enhancing the sweetness of a consumable comprising a carbohydrate sweetener, comprising adding one or more rebaudioside C polymorphs, especially rebaudioside C crystalline Form I, to the consumable in an amount effective to enhance the sweetness of the consumable. In one embodiment, the consumable has a sweetness intensity equivalent to about 5-12% (w/v-%) sucrose solution. In one embodiment, the consumable has a sweetness intensity equivalent to about 5% (w/v-%), about 6% (w/v-%), about 7% (w/v-%), or about 8% (w/v-%) sucrose solution. In one embodiment, the consumable has a sweetness intensity equivalent to about 9% (w/v-%), about 10% (w/v-%), about 11% (w/v-%), or about 12% (w/v-%) sucrose solution.

In one embodiment, one or more rebaudioside C polymorphs, especially rebaudioside C crystalline Form I, are added to the consumable in an amount to obtain a concentration of from about 150 μM to about 600 μM.

Additional embodiments and advantages of the invention will be set forth in part of the description that follows, and will flow from the description, or may be learned by practice of the invention. The embodiments and advantages of the invention will be realized and attained by means of the elements and combinations particularly pointed out in the appended claims.

It is to be understood that both the foregoing summary and the following detailed description are exemplary and explanatory only and are not restrictive of the invention, as claimed.

### BRIEF DESCRIPTION OF THE DRAWINGS

FIG. 1 is a powder x-ray diffraction scan comparing amorphous rebaudioside C (B2-orig powder), rebaudioside C crystallized from methanol/isopropanol (G1-MeOH/iPrOH), rebaudioside C crystallized from anhydrous methanol (D1-MeOH), rebaudioside C crystallized from absolute ethanol (A1-EtOH), and rebaudioside recrystallized from methanol/isopropanol (Combined and recrystallized from MeOH/iPrOH) as described in Example 1, on a plot of the scattering intensity versus d-spacing.

FIG. 2 is a powder x-ray diffraction scan of rebaudioside C crystalline Form I, on a plot of the scattering intensity versus d-spacing.

### DETAILED DESCRIPTION OF THE INVENTION

Rebaudioside C (hereinafter also “Reb C”) has the following chemical formula:

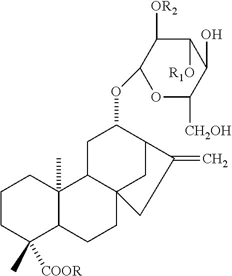

wherein R and R₁ are glucose and R₂ is rhamnose. Reb C can be prepared by methods known in the art, such as by isolating from Stevia rebaudiana plant material as described in U.S. Pat. No. 4,361,697, which is fully incorporated by reference herein in its entirety.

Reb C may contain one or more asymmetric centers and may thus give rise to enantiomers, diastereomers, and other stereoisomeric forms. The present invention is meant to encompass the uses of all such possible forms, as well as their racemic and resolved forms and mixtures thereof. The individual enantiomers may be separated according to methods known to those of ordinary skill in the art in view of the present disclosure. All tautomers are intended to be encompassed by the present invention as well.

As used herein, the term “stereoisomers” is a general term for all isomers of individual molecules that differ only in the orientation of their atoms in space. It includes enantiomers and isomers of compounds with more than one chiral center that are not mirror images of one another (diastereomers).

The term “chiral center” refers to a carbon atom to which four different groups are attached.

The terms “enantiomer” and “enantiomeric” refer to a molecule that cannot be superimposed on its mirror image and hence is optically active wherein the enantiomer rotates the plane of polarized light in one direction and its mirror image compound rotates the plane of polarized light in the opposite direction.

The term “racemic” refers to a mixture of equal parts of enantiomers and which mixture is optically inactive.

The term “resolution” refers to the separation or concentration or depletion of one of the two enantiomeric forms of a molecule.

The terms “a” and “an” refer to one or more.

As used herein, the term “sweetness intensity” refers to the relative strength of sweet sensation as observed or experienced by an individual, e.g., a human, or a degree or amount of sweetness detected by a taster, for example on the scale from 0 (none) to 8 (very strong) used in sensory evaluations according to the procedure described in American Society for Testing Materials, Special Technical Publication-434: “Manual on Sensory Testing Methods,” ASTM International, West Conshohocken, Pa. (1996).

As used herein, the phrase “sweet taste enhancing effect” means that the effect of Reb C is such that the sensory perception of the sweet flavor is potentiated in a more than additive manner, i.e., synergistically.

As used herein, the term “off-taste” refers to an amount or degree of taste that is not characteristically or usually found in a consumable. For example, an off-taste is an undesirable taste of a sweetened consumable to the consumers, such as, a bitter taste, a licorice-like taste, a metallic taste, an aversive taste, a nasty taste, an astringent taste, a delayed sweetness onset, and a lingering sweet aftertaste, and the like.

As used herein, the phrase “the detection threshold for its intrinsic sweetness” refers to the concentration of Reb C polymorph at which the sweetness of the Reb C polymorph is perceptible to an individual, e.g., a human.

As used herein in connection with a measured quantity, “about” refers to the normal variations in that measured quantity, as expected by the skilled artisan making the measurement and exercising a level of care commensurate with the objective of measurement and the precision of the measuring equipment.

As used herein, the term “w/v-%” refers to the weight of a component (in grams) for every 100 ml of the liquid composition of the present invention.

As used herein, the term “dry weight” or “by weight on dry basis” refers to the weight of a solid composition after all water content has been removed by drying the composition.

As used herein, the term “substantially” or “substantially pure” refers to a Reb C composition that includes at least about 90% by dry weight of Reb C, in another embodiment from about 90% to about 95% by dry weight of Reb C, and in yet another embodiment from about 99% to about 100% by dry weight of Reb C.

As used herein, the term “crystallization solution” refers to a liquid comprising one or more organic solvents. Non-limiting examples of organic solvents include alcohol, acetone, and acetonitrile. Alcohol, as used herein, refers to any straight, branched, or cyclic, substituted or unsubstituted alkyl, alkenyl, or alkynyl group attached to at least one hydroxyl moiety. Non-limiting examples of alcohols include ethanol, methanol, isopropanol, 1-propanol, 1-butanol, 2-butanol, tert-butanol, and isobutanol. The crystallization solution can also contain water.

As used herein, the term “slurry solution” refers to a liquid comprising one or more organic solvents, in which substantially pure Reb C is only sparingly soluble.

As used herein, the term “crude rebaudioside C solid” refers to a solid that includes at least 40% by dry weight of Reb C.

As used herein, the term “Stevia extract solid” refers to any solid extracted from the leaves of Stevia rebaudiana that includes from about 0.6% to about 80% by dry weight of Reb C.

As used herein, the terms “crystalline form” and “polymorph” are synonymous and refer to the ability of molecules within a solid material to exist in a specific orderly repeating pattern extending in all three spatial dimensions.

As used herein, the term “seed” refers to a small piece of a polycrystal material from which a large crystal of the same material can be grown. Typically, the large crystal can be grown by dipping the seed into a solution of the same material.

As used herein, the term “seeding” refers to the process of adding crystal seeds to a solution of the same material to grow larger crystals the molecular constituents of which are arranged within the larger crystals similarly, if not identically, to the molecular constituents of the crystal seeds.

As used herein, the term “minimal amount” refers to the smallest volume of solvent required to completely dissolve a solute to form a homogenous solution.

As used herein, the phrase “synergistically enhance the sweetness” means that the effect of Reb C or polymorphs of Reb C with a carbohydrate sweetener is such that the sensory perception of the sweet flavor is potentiated in a more than additive manner.

Unless otherwise specified, the phrase “carbohydrate sweetener” includes caloric sweeteners, such as, sucrose, fructose, glucose, high fructose corn syrup (containing fructose and glucose), xylose, arabinose, rhamnose, and sugar alcohols, such as erythritol, xylitol, mannitol, sorbitol, and inositol.

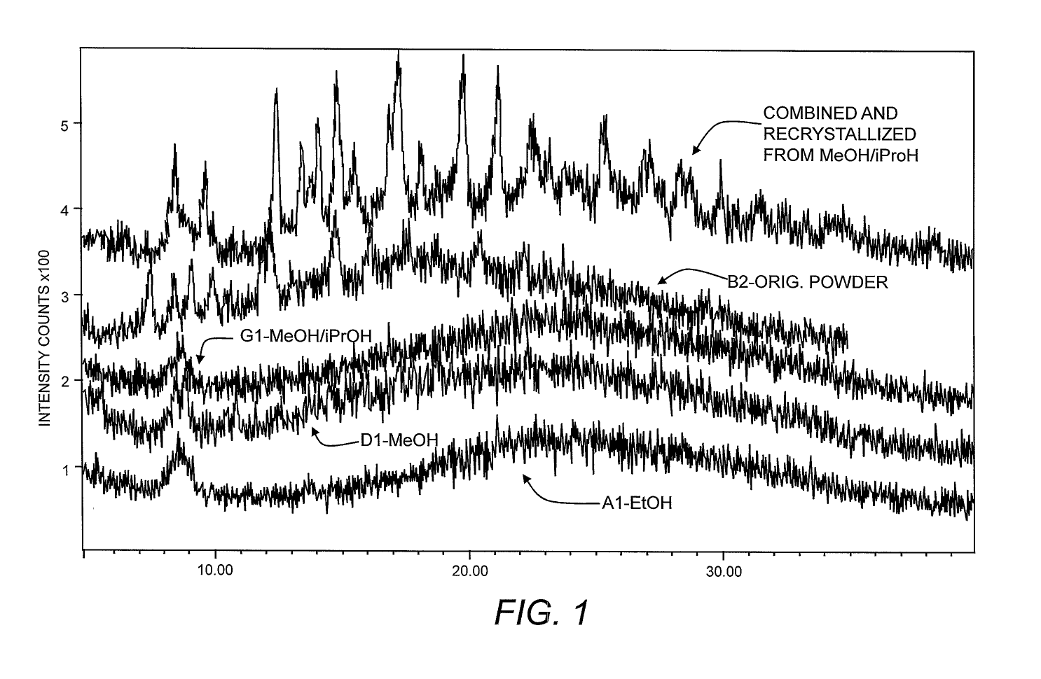

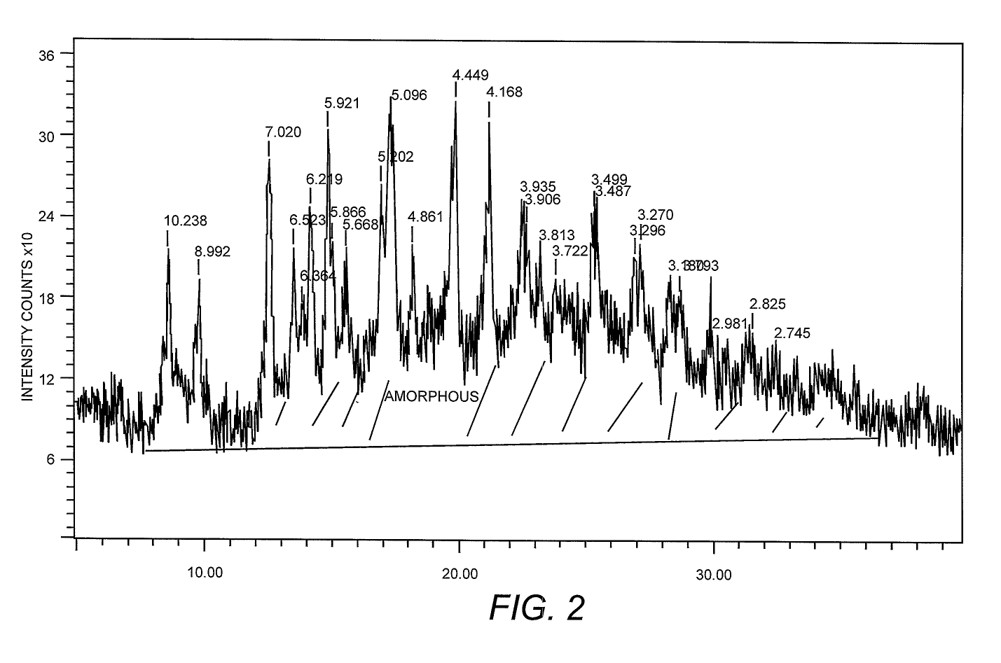

Exemplary embodiments of this invention provide a method for purifying Reb C to produce a substantially pure form of Reb C by crystallizing Reb C from a crystallization solution comprising an organic solvent. Other exemplary embodiments of this invention encompass compositions comprising one or more polymorphs of Reb C. Still other exemplary embodiments of this invention encompass methods of preparing polymorph forms of Reb C. Exemplary embodiments of this invention are described in detail below and illustrated in FIGS. 1 and 2.

### Methods for Purifying Reb C

Reb C (≧93% HPLC purity) can be obtained commercially as a byproduct of rebaudioside A (“Reb A”) purification and is largely produced through iterative cycles of separation and purification from Stevia extracts. Crude Stevia extracts comprising Reb C are also commercially available. However, these Stevia extracts comprise Reb A from about 40% to about 95% by dry weight, about 60% to about 85% by dry weight, or about 70% to about 85% by dry weight. In one embodiment, crude Reb C can be extracted from Stevia plants and be purified by crystallization or recrystallization. Primary impurities include other steviol glycosides, such as stevioside, Reb A, rebaudioside B (“Reb B”), and rebaudioside D (“Reb D”). Steviol glycoside impurities can be removed by varying the amount of water or organic solvent in a crystallization solution. Accordingly, the method of purification depends on the impurities present in the crude Reb C starting material.

One aspect of the invention is directed to a method for purifying Reb C. In one embodiment of the invention, a Reb C starting material can be combined with a crystallization solution to form a crude Reb C solution. In one embodiment, a Reb C starting material is a Stevia extract solid. In another embodiment, a Reb C starting material is a crude Reb C solid. In one embodiment, the crystallization solution comprises one or more organic solvents. In another embodiment, the crystallization solution comprises a mixture of water and one or more organic solvents. The crystallization solution can comprise water in an amount from about 5% to about 25% by weight and one or more organic solvents. Alternatively, the crystallization solution can comprise water in an amount from about 15% to about 20% by weight and one or more organic solvents.

In another embodiment, the crystallization solution comprises an alcohol, such as ethanol, methanol, propanol, isopropanol, or mixtures thereof. In another embodiment, the crystallization solution comprises absolute ethanol. In another embodiment, the crystallization solution comprises anhydrous methanol. In another embodiment, the crystallization solution comprises a mixture of isopropanol and methanol. In this aspect of the invention, isopropanol and methanol can be combined in the crystallization solution in a weight ratio ranging from about 15 parts to about 1 part isopropanol to about 1 part methanol. In another embodiment, isopropanol and methanol can be combined in the crystallization solution in a weight ratio from about 10 parts isopropanol to about 1 part methanol. In another embodiment, isopropanol and methanol can be combined in the crystallization solution in a weight ratio ranging from about 5 parts to about 1 part isopropanol to about 1 part methanol. In another embodiment, isopropanol and methanol can be combined in the crystallization solution in a weight ratio from about 2 parts isopropanol to about 1 part methanol.

In one embodiment, the crude Reb C solution comprises the crystallization solution and Reb C starting material in a weight ratio ranging from about 30 parts to about 1 part Reb C starting material to about 1 part crystallization solution. In another exemplary embodiment, the crude Reb C solution comprises the crystallization solution and Reb C starting material in a weight ratio ranging from about 20 parts to about 1 part Reb C starting material to about 1 part crystallization solution. In another embodiment, the crude Reb C solution comprises the crystallization solution and Reb C starting material in a weight ratio ranging from about 30 parts to about 1 part crystallization solution to about 1 part Reb C starting material.

In one embodiment, the method of purifying Reb C can be carried out at approximately room temperature. Room temperature is from about 20° C. to about 27° C. In one embodiment, the method can be carried out at 20° C. In another embodiment, the method further comprises the step of heating the crude Reb C solution. In another embodiment, the step of heating the crude Reb C solution comprises heating the crude Reb C solution to a temperature in a range from about 20° C. to about 70° C., from about 20° C. to about 60° C., from about 20° C. to about 40° C., or from about 40° C. to about 60° C. In another embodiment, the step of heating the crude Reb C solution comprises heating the crude Reb C solution to about reflux temperature. The step of heating the crude Reb C solution comprises heating the crude Reb C solution for about 0.25 hours to about 8 hours. In another exemplary embodiment, wherein the method for purifying Reb C comprises the step of heating the crude Reb C solution, the method further comprises the step of cooling the crude Reb C solution. In one embodiment, the step of cooling the crude Reb C solution comprises cooling the crude Reb C solution to a temperature in the range from about 4° C. to about 25° C. The step of cooling the crude Reb C solution comprises cooling the crude Reb C solution for about 0.5 hours to about 24 hours.

The method for purifying Reb C further comprises the step of crystallizing substantially pure Reb C from the crude Reb C solution to produce substantially pure Reb C crystals comprising Reb C in an amount greater than about 95% by weight on a dry basis, greater than about 97% by weight on a dry basis, greater than about 98% by weight on a dry basis, or greater than about 99% by weight on a dry basis. The crude Reb C solution can be stirred or left unstirred during the crystallization step.

In another embodiment, the method of crystallizing substantially pure Reb C can further comprise the optional step of seeding the crude Reb C solution at an appropriate temperature with substantially pure seed crystals of Reb C in an amount sufficient to promote crystallization. In a particular embodiment, isolated Reb C crystalline Form I crystals can be used to seed the crude Reb C solution at an appropriate temperature to promote crystallization of substantially pure Reb C crystalline Form I crystals. The amount of substantially pure Reb C seed crystals sufficient to promote crystallization comprises from about 0.0001% to about 1% by weight of Reb C present in the crude Reb C solution. In another embodiment, the amount of substantially pure Reb C seed crystals sufficient to promote crystallization comprises from about 0.01% to about 1% by weight of Reb C present in the crude Reb C solution. A suitable temperature for the step of seeding comprises a temperature in a range from about 5° C. to about 30° C. In one embodiment, suitable temperature ranges for the step of seeding include from about 10° C. to about 25° C., from about 15° C. to about 20° C., from about 5° C. to about 15° C., from about 15° C. to about 30° C., from about 10° C. to about 15° C., or from about 15° C. to about 20° C. In one embodiment, the suitable temperature for the step of seeding is room temperature. In one embodiment, the suitable temperature for the step of seeding is 20° C. In another embodiment, the suitable temperature for the step of seeding is 25° C.

In another embodiment, the method further comprises the steps of separating and washing the substantially pure Reb C crystals. The substantially pure Reb C crystals can be separated from the crude Reb C solution by a variety of solid-liquid separation techniques that utilize centrifugal force, that include, without limitation, vertical and horizontal perforated basket centrifuge, solid bowl centrifuge, decanter centrifuge, peeler type centrifuge, pusher type centrifuge, Heinkel type centrifuge, disc stack centrifuge and cyclone separation. Additionally, separation can be enhanced by any pressure, vacuum, or gravity filtration methods, that include without limitation, the use of belt, drum, nutsche type, leaf, plate, Rosenmund type, sparkler type, and bag filters and filter press. Operation of the Reb C solid-liquid separation device can be continuous, semi-continuous or in batch mode. The substantially pure Reb C crystals also can be washed on the separation device using various organic solvents and mixtures thereof. The substantially pure Reb C crystals can be partially or totally dried on the separation device using any number of gases, including, without limitation, nitrogen or argon, to evaporate residual liquid solvent. The substantially pure Reb C crystals can be automatically or manually removed from the separation device using liquids, gases or mechanical means by either dissolving the solid or maintaining the solid form.

In still another embodiment, the method further comprises the step of drying the substantially pure Reb C crystals. Such methods are known to those skilled in the art and include, but are not limited to, the use of a rotary vacuum dryer, fluid bed dryer, rotary tunnel dryer, plate dryer, tray dryer, Nauta type dryer, spray dryer, flash dryer, micron dryer, pan dryer, high and low speed paddle dryer and microwave dryer. In an exemplary embodiment, the step of drying comprises drying the substantially pure Reb C crystals using a nitrogen or argon purge to remove the residual solvent at a temperature in a range from about 40° C. to about 60° C. for about 5 hours to about 10 hours.

In yet another embodiment, wherein the crude Reb C solution comprises substantially no Reb A impurity, the method further comprises the step of slurrying the substantially pure Reb C crystals with a slurry solution prior to the step of drying the substantially pure Reb C crystals. In another embodiment, wherein the crude Reb C solution comprises substantially no Reb D impurity, the method further comprises the step of slurrying the substantially pure Reb C crystals with a slurry solution prior to the step of drying the substantially pure Reb C crystals. The slurry can be a mixture comprising a solid and a slurry solution comprising an organic solvent, wherein the solid comprises the substantially pure Reb C crystals and is only sparingly soluble in the slurry solution. In another embodiment, the substantially pure Reb C crystals and slurry solution can be present in the slurry in a weight ratio ranging from about 15 parts to about 1 part slurry solution to about 1 part substantially pure Reb C crystals. In one embodiment, the slurry can be maintained at room temperature. In another embodiment, the step of slurrying comprises heating the slurry to a temperature in a range from about 20° C. to about 40° C. The substantially pure Reb C crystals can be slurried for about 0.5 hours to about 24 hours.

In still yet another embodiment, the method further comprises the steps of separating the substantially pure Reb C crystals from the slurry solution of the slurry and washing the substantially pure Reb C crystals followed by the step of drying the substantially pure Reb C crystals.

If further purification is desired, the method of purifying Reb C described herein may be repeated or the substantially pure Reb C crystals may be further purified using an alternative purification method.

In a more specific embodiment of the invention, the method of purifying Reb C comprises the steps of: (a) supplying a Stevia extract solid or a crude Reb C solid, wherein the Stevia extract solid contains at least 0.6% of Reb C by dry weight and the crude Reb C solid contains at least 40% of Reb C by dry weight, (b) adding a crystallization solution to the Stevia extract solid or crude Reb C solid of step (a) to produce a crude Reb C solution, (c) seeding the crude Reb C solution with isolated Reb C crystalline Form I crystals, (d) allowing the crude Reb C solution to dry completely at room temperature, (e) recovering the Reb C crystals formed in step (d), (f) adding a crystallization solution comprising methanol and isopropanol to the Reb C crystals of step (e) to completely dissolve the crystals, (g) allowing the solution of step (f) to dry completely at room temperature, and (h) recovering the isolated Reb C crystalline Form I crystals formed in step (g). In another embodiment of the invention, the crystallization solution of step (b) comprises acetone, acetonitrile, methanol, ethanol, propanol, isopropanol, butanol, 2-butanol, tert-butanol, or mixtures thereof. In another embodiment, the crystallization solution of step (b) comprises one or more alcohols and water. In another embodiment, the crystallization solution of step (b) comprises ethanol. In another embodiment, the crystallization solution of step (b) comprises methanol. In another embodiment, the crystallization solution of step (b) comprises isopropanol. In a further embodiment, the crystallization solution of step (b) comprises both methanol and isopropanol. In yet a further embodiment, methanol and isopropanol present in the crystallization solution are in a weight ratio from about 5 parts to about 1 part isopropanol to about 1 part methanol. In another embodiment, methanol and isopropanol present in the crystallization solution are in a weight ratio from about 2 parts isopropanol to about 1 part methanol. In another embodiment, isopropanol and methanol can be combined in the crystallization solution in a weight ratio ranging from about 15 parts to about 1 part isopropanol to about 1 part methanol. In another embodiment, isopropanol and methanol can be combined in the crystallization solution in a weight ratio from about 10 parts isopropanol to about 1 part methanol. In another embodiment, the method further comprises seeding the crystallization solution of step (f) with isolated Reb C crystalline Form I crystals. In one embodiment, Reb C crystals dissolved in step (f) can be a mixture of crystals comprising two or more of the following: Reb C crystals crystallized from absolute ethanol, Reb C crystals crystallized from anhydrous methanol, and Reb C crystals crystallized from methanol/isopropanol.

In yet another specific embodiment of the invention, the method for purifying Reb C comprises the steps of: (a) supplying a Stevia extract solid or a crude Reb C solid, wherein the Stevia extract contains at least 0.6% of Reb C by dry weight and the crude Reb C solid contains at least 40% of Reb C by dry weight, (b) adding a crystallization solution comprising methanol and isopropanol to the Stevia extract solid or crude Reb C solid of step (a) to produce a crude Reb C solution, (c) seeding the crude Reb C solution of step (b) with isolated Reb C crystalline Form I crystals, (d) allowing the crude Reb C solution to dry completely at room temperature, and (e) recovering the isolated Reb C crystalline Form I crystals formed in step (d). In one embodiment, the method further comprises the step of heating the crude Reb C solution of step (b). In another embodiment, the method further comprises the steps of heating then cooling the crude Reb C solution of step (b). In another embodiment, the crude Reb C solution is stirred. In yet another embodiment, the method further comprises the steps of separating and washing the isolated Reb C crystalline Form I crystals. In another embodiment, the method further comprises the step of drying the isolated Reb C crystalline Form I crystals. In another embodiment, the crude Reb C solid comprises substantially no Reb A impurity and the method further comprises slurrying the isolated Reb C crystalline Form I crystals in a slurry solution. In another embodiment, the crude Reb C solid comprises substantially no Reb D impurity and the method further comprises slurrying the isolated Reb C crystalline Form I crystals in a slurry solution. In another embodiment, isopropanol and methanol can be combined in the crystallization solution of step (b) in a weight ratio ranging from about 15 parts to about 1 part isopropanol to about 1 part methanol. In another embodiment, isopropanol and methanol can be combined in the crystallization solution of step (b) in a weight ratio from about 10 parts isopropanol to about 1 part methanol.

### Methods for Crystallizing Reb C and Reb C Polymorphs

Reb C can be crystallized from absolute ethanol, anhydrous methanol and methanol/isopropanol as described herein. The crystallization of Reb C using the method described herein results in the formation of at least one new polymorph of Reb C (i.e., isolated Reb C crystalline Form I). Those of ordinary skill in the art will appreciate that both the crystallization solution and the temperatures of the crystallization process described herein may influence the resulting polymorphs of a substantially pure Reb C composition.

One aspect of the invention provides a method for making isolated Reb C crystalline Form I, comprising: (a) supplying a substantially pure Reb C solid, (b) adding a crystallization solution to the substantially pure Reb C solid of step (a) to completely dissolve the solid, (c) allowing the crystallization solution of step (b) to evaporate completely at room temperature, (d) recovering isolated Reb C crystals formed in step (c), (e) adding a crystallization solution comprising methanol and isopropanol to the Reb C crystals of step (d) to completely dissolve the crystals, (f) allowing the solution of step (e) to dry completely at room temperature, and (g) recovering isolated Reb C crystalline Form I crystals formed in step (e). In one embodiment, the crystallization solution of step (b) comprises acetone, acetonitrile, methanol, ethanol, propanol, isopropanol, butanol, 2-butanol, tert-butanol, or mixtures thereof. In another embodiment, the crystallization solution of step (b) comprises one or more alcohols and water. In another embodiment, the crystallization solution of step (b) comprises ethanol. In another embodiment, the crystallization solution of step (b) comprises methanol. In another embodiment, the crystallization solution of step (b) comprises isopropanol. In a further embodiment, the crystallization solution of step (b) comprises methanol and isopropanol. In yet a further embodiment, the methanol and isopropanol present in the crystallization solution are in a weight ratio from about 5 parts to about 1 part isopropanol to about 1 part methanol. In another embodiment, the methanol and isopropanol present in the crystallization solution are in a weight ratio from about 2 parts isopropanol to about 1 part methanol. In another embodiment, isopropanol and methanol can be combined in the crystallization solution in a weight ratio ranging from about 15 parts to about 1 part isopropanol to about 1 part methanol. In another embodiment, isopropanol and methanol can be combined in the crystallization solution in a weight ratio from about 10 parts isopropanol to about 1 part methanol. In one embodiment, Reb C crystals dissolved in step (e) can be a mixture of crystals comprising two or more of the following: Reb C crystals crystallized from absolute ethanol, Reb C crystals crystallized from anhydrous methanol, and Reb C crystals crystallized from methanol/isopropanol. In another embodiment, in step (e), the Reb C crystals of step (d) are first dissolved in a minimal amount of methanol to produce a Reb C/methanol solution which is then diluted with isopropanol at a volume ratio of 1:9. In another embodiment, the method further comprises seeding the crystallization solution of step (e) with isolated Reb C crystalline Form I crystals.

Another aspect of the invention provides for a method for making isolated Reb C crystalline Form I, comprising: (a) supplying a substantially pure Reb C solid, (b) adding a crystallization solution comprising methanol and isopropanol to the substantially pure Reb C solid of step (a) to completely dissolve the solid, (c) allowing the solution of step (b) to dry completely at room temperature, and (d) recovering isolated Reb C crystalline Form I crystals formed in step (c). In one embodiment of the invention, the substantially pure Reb C solid and the crystallization solution are combined in step (b) in a weight ratio from about 30 parts to about 1 part substantially pure Reb C to about 1 part crystallization solution. In another embodiment of the invention, the substantially pure Reb C solid and the crystallization solution are combined in step (b) in a weight ratio from about 30 parts to about 1 part crystallization solution to about 1 part substantially pure Reb C. In another embodiment, in step (b), the Reb C solid of step (a) is first dissolved in a minimal amount of methanol to produce a Reb C/methanol solution which is then diluted with isopropanol at a volume ratio of 1:9. In another embodiment, the method further comprises seeding the crystallization solution of step (b) with isolated Reb C crystalline Form I crystals.

Polymorphism is defined as the ability of a substance to exist in two or more crystalline states that have different arrangements and/or conformations of the molecules in the crystal lattice. Approximately 30% of organic compounds are believed to exhibit polymorphism (Zell, et al., Tetrahedron 56(36):6603-16 (2000)). Polymorphism is important in the formulation of pharmaceuticals, pigments and dyes, sweeteners, explosives, and agrochemicals. Polymorphism may cause physical properties such as density, melting point, and rate of dissolution to change.

Reb C crystalline Form I was identified by analysis of samples with powder x-ray diffraction (XRPD), a technique well known to those skilled in the art. FIGS. 1 are 2 are XRPD scans of substantially pure Reb C compositions obtained from the crystallization processes described herein. The XRPD scans of Reb C polymorphs were created by plotting the scattering intensity versus d-spacing. Samples can be analyzed by XRPD using a Shimadzu XRD-6000 X-ray powder diffractometer using Cu Kα (1.54 Å) radiation. The instrument is equipped with a long fine focus X-ray tube. Typically, the tube voltage and amperage can be set to 40 kV and 40 mA, respectively. The divergence and scattering slits can be set at 1°, and the receiving slit can be set at 0.15 mm. Diffracted radiation can be detected by a Shimadzu SC-1001 scintillation detector. A θ-2θ continuous scan at 3°/min (0.4 sec/0.02° step) from 2.5 to 40° 20 can be used. A silicon standard can be analyzed to check the instrument alignment. Data can be collected and analyzed using XRD-6000 v. 4.1.

FIG. 1 highlights the structural differences between amorphous Reb C (B2-orig powder), Reb C crystallized from methanol/isopropanol (G1-MeOH/iPrOH), Reb C crystallized from anhydrous methanol (D1-MeOH), Reb C crystallized from ethanol (A1-EtOH), and Reb C recrystallized from methanol/isopropanol (Combined and recrystallized from MeOH/iPrOH) as described in Example 1.

FIG. 2 shows a representative pattern for the Reb C crystalline Form I. In one embodiment of the invention, Reb C crystalline Form I has an XRPD pattern at Cu Kα wavelength 1.54 Å as shown in FIG. 2 and is further characterized by d-spacing distances (A) of significant peaks at: 8.6, 9.8, 12.6, 13.6, 13.9, 14.2, 14.9, 15.6, 17.0, 17.4, 18.2, 19.9, 21.3, 22.6, 23.3, 25.5, 27.2, 28.4, 28.9, and 30.0.

As illustrated in FIG. 1, the type of polymorph formed may be dependent on factors such as the composition of the crystallization solution, the temperature of the crystallization step, and the temperature during the drying/evaporation step.

Those of ordinary skill in the art should appreciate that the Reb C composition described herein can be modified to obtain a desired mixture of Reb C polymorphic and amorphous forms depending on the desired qualities of the Reb C composition (i.e., rate of dissolution, etc.). Those of ordinary skill in the art should also appreciate that the rate of dissolution of a composition may be important in the formulation of solid and liquid consumable compositions, non-limiting examples of which include chewing gum, baked goods, and beverages. In one embodiment, a substantially pure Reb C composition can comprise a particular polymorphic or amorphous form of Reb C in an amount in the range of about 1% to about 100% by weight. In a particular embodiment, a substantially pure Reb C composition comprises isolated Reb C crystalline Form I in an amount in the amount of about 1% to about 100% by weight. For example, a substantially pure Reb C composition can comprise isolated Reb C crystalline Form I in an amount greater than about 25% by weight, more particularly in an amount greater than about 50% by weight, still more particularly in an amount greater than about 75% by weight, or still even more particularly in an amount greater than about 85% by weight. Suitable amounts of Reb C polymorphic or amorphous forms also can be used within these ranges. In another embodiment, a substantially pure Reb C composition can comprise a combination of particular polymorphic and/or amorphous form of Reb C.

### Diffractometer

X-ray diffractometers useful in characterizing Reb C polymorphs of the invention can consist of a source of radiation, a monochromator to choose the wavelength, slits to adjust the shape of the radiation bean, a goniometer and a detector. Non-limiting examples of diffractometers that can be used include the Shimadzu XRD-6000 (Shimadzu Scientific Instruments 7102 Riverwood Drive, Columbia, Md., 21046 USA), Rigaku Ultima IV (Rigaku, 9009 New Trails Drive, The Woodlands, Texas, USA 77381), and X'Pert PRO MPD diffractometers (PANalytical Inc. 117 Flanders Road, Westborough, Mass. 01581 USA).

### Detector

When diffractometers are not equipped with built-in detectors, external detectors can be fitted for data acquisition. Non-limiting examples of detectors that can be used include the D/teX Ultra (Rigaku, 9009 New Trails Drive, The Woodlands, Texas, USA 77381) and X'Celerator detection systems (PANalytical Inc. 117 Flanders Road, Westborough, Mass. 01581 USA).

### Software

A number of software programs are available for data collection and analysis. Non-limiting examples of software useful in analyzing XRPD data include TREOR (Werner, P.-E. et al., J. Appl. Cryst. 18:365-370 (1985)), Crystallographica Search-Match (Oxford Cryosystems Ltd, 3 Blenheim Office Park, Lower Road, Long Hanborough Oxford OX29 8LN, United Kingdom), Jade (Jade, Materials Data, Inc., 1224 Concannon Blvd., Livermore, Calif. 94550, USA), and RayfleX (GE Inspection Technologies, GmbH, Robert-Bosch-Str. 3, 50354 Huerth, Germany)

### Reb C Polymorph Compositions

Reb C polymorphs can be used in combination with Reb A and/or dulcoside A in consumables, e.g., in food products, pharmaceuticals, dietary supplements, nutraceuticals, dental hygienic compositions, or other products as sweetness enhancers, which retain a desired sweetness but contain lower amounts of a carbohydrate sweetener, such as sucrose, glucose and fructose. In one embodiment, the present invention provides a consumable, comprising an effective amount of one or more Reb C polymorphs and a carbohydrate sweetener in a reduced amount in order to achieve the same level of sweetness when the carbohydrate sweetener is used alone in the traditional amount. By way of brief example, a common carbonated cola beverage can contain about 20 to 30 grams of sugar (e.g., fructose) and about 100 calories per 8 ounce serving. The present invention enables one to prepare a similar cola beverage with substantially reduced sugar and caloric content with the same level of sweetness. Reb C polymorphs enhance the sweet taste produced by the reduced sugar content, thereby creating an enhanced sweet taste based on the level of the sugar, without exhibiting any off-taste.

Suitable carbohydrate sweeteners of the present invention include, but are not limited to, sucrose, fructose, glucose, high fructose corn syrup (containing fructose and glucose), xylose, arabinose, rhamnose, and sugar alcohols, such as erythritol, xylitol, mannitol, sorbitol, or inositol. In one embodiment of the present invention, the carbohydrate sweetener is sucrose, fructose, glucose, high fructose corn syrup, xylose, arabinose or rhamnose, preferably sucrose, fructose, or glucose. In one aspect of this embodiment, the carbohydrate sweetener is sucrose. In another aspect of this embodiment, the carbohydrate sweetener is glucose. In another aspect of this embodiment, the carbohydrate sweetener is fructose. In another embodiment, the carbohydrate sweetener is a sugar alcohol.

Sucrose, also known as table sugar or saccharose, is a disaccharide of glucose and fructose. Its systematic name is α-D-glucopyranosyl-(1→2)-β-D-fructofuranose. Fructose and glucose are monosaccharide sugars.

In the consumables, one or more Reb C polymorphs are used in an amount effective to enhance the sweetness of a carbohydrate sweetener without exhibiting any off-taste. Any amount of one or more Reb C polymorphs that provide the desired degree of sweetness enhancement can be used. In one embodiment, the concentration at which one or more Reb C polymorphs are used in the present invention is at, slightly above, or below the detection threshold for its intrinsic sweetness. In one embodiment, one or more Reb C polymorphs are present in the consumable of the present invention at a concentration of from about 150 μM to about 600 μM. In one embodiment, one or more Reb C polymorphs are present in the consumable of the present invention at a concentration of from about 150 μM to about 350 μM. In one embodiment, one or more Reb C polymorphs are present in the consumable of the present invention at a concentration of from about 250 μM to about 350 μM. In one embodiment, one or more Reb C polymorphs are present in the consumable of the present invention at a concentration of from about 350 μM to about 600 μM. In one embodiment, one or more Reb C polymorphs are in the consumable of the present invention at a concentration of about 150 μM, about 160 μM, about 170 μM, about 180 μM, about 190 μM, about 200 μM, about 210 μM, about 220 μM, about 230 μM, about 240 μM, about 250 μM, about 260 μM, about 270 μM, about 280 μM, about 290 μM, about 300 μM, about 310 μM, about 320 μM, about 330 μM, about 340 μM, or about 350 μM. In one embodiment, one or more Reb C polymorphs are present in the consumable of the present invention at a concentration of about 360 μM, about 370 μM, about 380 μM, about 390 μM, about 400 μM, about 410 μM, about 420 μM, about 430 μM, about 440 μM, about 450 μM, about 460 μM, about 470 μM, about 480 μM, about 490 μM, about 500 μM, about 510 μM, about 520 μM, about 530 μM, about 540 μM, about 550 μM, about 560 μM, about 570 μM, about 580 μM, about 590 μM, or about 600 μM. Useful concentrations of one or more Reb C polymorphs in the consumable of the present invention include about 250 μM or about 300 μM, and specifically 300 μM. In one embodiment, the ratio of one or more Reb C polymorphs to sucrose is approximately from 1:150 to 1:200 in a solid consumable. In one embodiment, the consumable of the present invention contains about 0.1 to 0.5 g, preferably about 0.3 g, of one or more Reb C polymorphs for every 50 to 100 g of the carbohydrate sweetener.

U.S. Prov. Appl. Nos. 61/179,330 and 61/226,679, filed May 18, 2009 and Jul. 17, 2009, respectively, relate to the use of Reb C, or a stereoisomer thereof, for enhancing the sweet taste of carbohydrate sweeteners. U.S. Prov. Appl. Nos. 61/179,330 and 61/226,679 are fully incorporated by reference herein in their entirety. Reb C polymorphs can similarly be useful for enhancing the sweetness of a consumable having a sweetness intensity equivalent to about 5-12% (w/v-%) sucrose solution. In this aspect of the invention, the consumable is preferably a sweet juice or a soft drink having a sweetness intensity equivalent to about 5-12% (w/v-%) sucrose solution. One or more Reb C polymorphs can be added to this consumable having a sweetness intensity equivalent to about 5-12% (w/v-%) sucrose solution by admixing with the consumable or admixing with a component of the consumable. In one embodiment, one or more Reb C polymorphs are added to a consumable having a sweetness intensity equivalent to about 5% (w/v-%), about 6% (w/v-%), about 7% (w/v-%), or about 8% (w/v-%) sucrose solution to enhance the sweetness of the consumable. In one embodiment, one or more Reb C polymorphs are added to a consumable having a sweetness intensity equivalent to about 9% (w/v-%), about 10% (w/v-%), about 11% (w/v-%), or about 12% (w/v-%) sucrose solution to enhance the sweetness of the consumable. In one embodiment, the sweetness intensity of the consumable of the present invention containing one or more Reb C polymorphs is equivalent to about 5-7% (w/v-%) sucrose solution. In another embodiment, the sweetness intensity of the consumable of the present invention containing one or more Reb C polymorphs is equivalent to about 8-12% (w/v-%) sucrose solution. In one embodiment, the sweetness intensity of the consumable of the present invention containing one or more Reb C polymorphs is equivalent to about 5% (w/v-%), about 6% (w/v-%), about 7% (w/v-%), about 8% (w/v-%), about 9% (w/v-%), about 10% (w/v-%), about 11% (w/v-%), or about 12% (w/v-%) sucrose solution.

Consumables include all food products, dietary supplements, nutraceuticals, pharmaceutical compositions, dental hygienic compositions, and cosmetic products. Also, one or more sweeteners other than carbohydrate sweeteners can be present in the consumables of the present invention. The carbohydrate sweetener can be present in the consumable inherently (e.g., in food products containing fruits) or the carbohydrate sweetener is added into the consumable.

The phrase “food product” as used herein includes, but is not limited to, fruits, vegetables, juices, meat products such as ham, bacon and sausage; egg products, fruit concentrates, gelatins and gelatin-like products such as jams, jellies, preserves, and the like; milk products such as ice cream, sour cream and sherbet; icings, syrups including molasses; corn, wheat, rye, soybean, oat, rice and barley products, nut meats and nut products, cakes, cookies, confectionaries such as candies, gums, fruit flavored drops, and chocolates, chewing gum, mints, creams, icing, ice cream, pies and breads, beverages such as coffee, tea, carbonated soft drinks, such as COKE® and PEPSI®, non-carbonated soft drinks, juices and other fruit drinks, sports drinks such as GATORADE®, coffee, teas, iced teas, cola, alcoholic beverages, such as beers, wines and liquors, and KOOL-AID®. Preferably, the food products in which the sweetness of the carbohydrate sweetener is enhanced with one or more Reb C polymorphs contains a decreased level of the carbohydrate sweetener. For example, an improved carbonated soft drink can be produced with the same sweetness as the known carbonated soft drink but with a lower sugar content by adding one or more Reb C polymorphs.

Food products also include condiments such as herbs, spices and seasonings, flavor enhancers, such as monosodium glutamate. A food product also includes prepared packaged products, such as dietetic sweeteners, liquid sweeteners, granulated flavor mixes which upon reconstitution with water provide non-carbonated drinks, instant pudding mixes, instant coffee and tea, coffee whiteners, malted milk mixes, pet foods, livestock feed, tobacco, and materials for baking applications, such as powdered baking mixes for the preparation of breads, cookies, cakes, pancakes, donuts and the like. Food products also include diet or low-calorie food and beverages containing little or no sucrose. Especially preferred food products are carbonated beverages containing one or more Reb C polymorphs. Other examples of food products envisioned in accordance with the present invention are described below and throughout the specification.

In another embodiment, the food product is selected from the group consisting of fruits, vegetables, juices, meat products such as ham, bacon and sausage; egg products, fruit concentrates, gelatins and gelatin-like products such as jams, jellies, preserves, and the like; milk products such as ice cream, sour cream and sherbet; icings, syrups including molasses; corn, wheat, rye, soybean, oat, rice and barley products, nut meats and nut products, cakes, cookies, confectionaries such as candies, gums, fruit flavored drops, and chocolates, creams, icing, ice cream, pies and breads.

In one embodiment, the invention is directed to a method of decreasing the amount of a carbohydrate sweetener in a consumable, such as a food product or a pharmaceutical composition, to exhibit a given level of sweetness, wherein the method comprises reducing the amount of the carbohydrate sweetener and adding one or more Reb C polymorphs in an amount effective to maintain the given level of sweetness of the consumable.

In one embodiment, the food product is a beverage or a drink comprising a carbohydrate sweetener and one or more Reb C polymorphs. Examples of suitable beverages in which having a sweet taste is desired include, but are not limited to coffee, teas, such as black tea, green tea, fermented tea, semi-fermented tea, carbonated soft drinks, such as COKE® and PEPSI®, non-carbonated soft drinks, lemonade, juices and other fruit drinks, sports drinks, such as GATORADE®, iced teas, cola, alcoholic beverages, such as beers, wines and liquors, and KOOL-AID®. In one embodiment, one or more Reb C polymorphs are present at a concentration of from about 150 μM to about 600 μM. In certain embodiments, one or more Reb C polymorphs are present at a concentration of from about 150 μM to about 350 μM. In one embodiment, one or more Reb C polymorphs are present at a concentration of from about 250 μM to about 350 μM. In one embodiment, one or more Reb C polymorphs are present at a concentration of from about 350 μM to about 600 μM. In one embodiment, one or more Reb C polymorphs are present in the beverage or drink at a concentration of about 150 μM, about 160 μM, about 170 μM, about 180 μM, about 190 μM, about 200 μM, about 210 μM, about 220 μM, about 230 μM, about 240 μM, about 250 μM, about 260 μM, about 270 μM, about 280 μM, about 290 μM, about 300 μM, about 310 μM, about 320 μM, about 330 μM, about 340 μM, or about 350 μM. In one embodiment, one or more Reb C polymorphs are present in the consumable of the present invention at a concentration of about 360 μM, about 370 μM, about 380 μM, about 390 μM, about 400 μM, about 410 μM, about 420 μM, about 430 μM, about 440 μM, about 450 μM, about 460 μM, about 470 μM, about 480 μM, about 490 μM, about 500 μM, about 510 μM, about 520 μM, about 530 μM, about 540 μM, about 550 μM, about 560 μM, about 570 μM, about 580 μM, about 590 μM, or about 600 μM. Useful concentrations of one or more Reb C polymorphs in the beverage or drink of the present invention is about 250 μM or about 300 μM, and specifically 300 μM. In one embodiment, the beverage or drink comprises one carbohydrate sweetener. In another embodiment, it comprises more than one carbohydrate sweetener. In certain embodiments, the beverage or drink comprises sucrose and corn syrup, or it comprises sucrose and aspartame as sweeteners.

One embodiment of the invention is directed to a method of enhancing the sweet taste of a cola beverage, such as COKE® or PEPSI®, comprising administering to a subject a cola drink, comprising a carbohydrate sweetener and one or more Reb C polymorphs in an amount to enhance the sweet taste of the carbohydrate sweetener without exhibiting any off-taste. In a preferred embodiment, the cola beverage contains a reduced amount of sugar but maintains substantially the original level of sweet taste.

Cola beverages are prepared by mixing cola concentrate with carbonated water. Typically about 50 mL of cola concentrate is added per 250 mL of carbonated water. Cola concentrate can be prepared by mixing cola flavor, caramel color, and optionally caffeine with water, one or more carbohydrate sweeteners, one or more Reb C polymorphs, and one or more acid components.

A cola flavor refers to either a natural or artificial flavor. Such cola flavors are commercially available. Commercial cola flavors are available, for example, from International Flavor and Fragrances, Dayton, N.J.; Artificial—#13573011 and Natural #K3559549. Commercial cola flavors are also available from Tastemaker, Cincinnati, Ohio, and Givaudan Roure, Clifton, N.J.

The acid component refers to an ingredient that contributes sourness to the beverage and is added to balance the flavor profile. Acids include malic acid, citric acid, phosphoric acid or combinations thereof.

For example, the cola concentrate can be prepared by mixing phosphoric acid (75% Rhone-Poulenc), citric acid (anhydrous, ADM, Decatur, Ill.), caffeine (Mallinckrodt, Paris, Ky.), caramel Color (DS400, Sethness, Chicago, Ill.), cola Flavor (SN018976, International Flavors and Fragrances, Dayton, N.J.), sucrose, one or more Reb C polymorphs, and water. The concentrate is blended until all ingredients are dissolved (30-40 minutes) using a magnetic stirring plate. Fifty milliliters of the concentrate are added to 250 mL of carbonated water to complete the preparation of the cola beverage. Fifty milliliters of cola concentrate typically contains from 0.01 to 5 mL of phosphoric acid, preferably about 0.01-1 mL, 0.1 to 100 g of sucrose, preferably about 1-10 g, about 0.1 to 0.5 g of one or more Reb C polymorphs, preferably about 0.3 g of one or more Reb C polymorphs, for every 50 to 100 g of sucrose, about 0.001 g to 0.1 g of citric acid, preferably about 0.005-0.1 g, 0.001 to 1 g of caffeine, preferably about 0.01 to 0.1 g of caffeine, 0.01 to 5 g of caramel flavor, preferably about 0.05 to 1 g, 0.001 to about 10 mL of cola flavor, preferably about 0.01 to about 2 mL.

In certain embodiments, the improved food product, such as the cola beverage, e.g., COKE® or PEPSI®, contains a reduced amount of sugar compared to the prior art cola beverage. The method can be performed such that the amount of sugar required to maintain the desired sweetness of the cola beverage is reduced by at least about 10%, 20%, 30%, 40%, 50%, 60%, 70%, 80%, 90%, or 95%, or from about 60% to about 99%, or alternatively from about 20% to about 50%. Thus, in a more specific embodiment, the cola beverage comprising a carbohydrate sweetener and one or more Reb. C polymorphs, contains Reb C polymorphs in an amount sufficient to reduce the amount of sugar required to maintain the desired sweetness of the beverage by 10%, 20%, 30%, 40%, 50%, 60%, 70%, 80%, 90%, or 95%, or from about 60% to about 99%, or alternatively from about 30% to about 70%. Of course, in other embodiments, the amount of sugar required can be decreased to differing extents.

Food products of the present invention also include animal food products, comprising a carbohydrate sweetener and one or more Reb C polymorphs in an amount sufficient to enhance the sweet taste of the carbohydrate sweetener without exhibiting any off-taste. Animal food products are well known in the art, see, e.g., U.S. Pat. No. 6,403,142, and include dog food, cat food, rabbit food, and the like. The animal food product also include food products useful for feeding livestock, such as cattle, bison, pigs, chicken, and the like. In another embodiment, the animal food product of the present invention is a solid hypoallergenic pet food, comprising a component that contains protein or protein fragments wherein all of said component is partially hydrolyzed and further comprises Reb C polymorphs. In certain embodiments, one or more Reb C polymorphs are present in the animal food product in an amount as described above for food products.

In one embodiment, the consumable is a pharmaceutical composition comprising a carbohydrate sweetener and one or more Reb C polymorphs. Preferred compositions are pharmaceutical compositions comprising one or more Reb C polymorphs and one or more pharmaceutically acceptable excipients. These pharmaceutical compositions can be used to formulate pharmaceutical drugs containing one or more active agents that exert a biological effect other than sweetness enhancement. The pharmaceutical composition preferably further comprises one or more active agents that exert a biological effect. Such active agents include pharmaceutical and biological agents that have an activity other than taste enhancement. Such active agents are well known in the art. See, e.g., The Physician's Desk Reference. Such compositions can be prepared according to procedures known in the art, for example, as described in Remington's Pharmaceutical Sciences, Mack Publishing Co., Easton, Pa., USA. In one embodiment, such an active agent includes bronchodilators, anorexiants, antihistamines, nutritional supplements, laxatives, analgesics, anesthetics, antacids, H₂-receptor antagonists, anticholinergics, antidiarrheals, demulcents, antitussives, antinauseants, antimicrobials, antibacterials, antifungals, antivirals, expectorants, anti-inflammatory agents, antipyretics, and mixtures thereof. In one embodiment, the active agent is selected from the group consisting of antipyretics and analgesics, e.g., ibuprofen, acetaminophen, or aspirin; laxatives, e.g., phenolphthalein dioctyl sodium sulfosuccinate; appetite depressants, e.g., amphetamines, phenylpropanolamine, phenylpropanolamine hydrochloride, or caffeine; antacidics, e.g., calcium carbonate; antiasthmatics, e.g., theophylline; antidiuretics, e.g., diphenoxylate hydrochloride; agents active against flatulence, e.g., simethecon; migraine agents, e.g., ergotaminetartrate; psychopharmacological agents, e.g., haloperidol; spasmolytics or sedatives, e.g., phenobarbitol; antihyperkinetics, e.g., methyldopa or methylphenidate; tranquilizers, e.g., benzodiazepines, hydroxinmeprobramates or phenothiazines; antihistaminics, e.g., astemizol, chloropheniramine maleate, pyridamine maleate, doxlamine succinate, bromopheniramine maleate, phenyltoloxamine citrate, chlorocyclizine hydrochloride, pheniramine maleate, and phenindamine tartrate; decongestants, e.g., phenylpropanolamine hydrochloride, phenylephrine hydrochloride, pseudoephedrine hydrochloride, pseudoephedrine sulfate, phenylpropanolamine bitartrate, and ephedrine; beta-receptor blockers, e.g., propanolol; agents for alcohol withdrawal, e.g., disulfuram; antitussives, e.g., benzocaine, dextromethorphan, dextromethorphan hydrobromide, noscapine, carbetapentane citrate, and chlophedianol hydrochloride; fluorine supplements, e.g., sodium fluoride; local antibiotics, e.g., tetracycline or cleocine; corticosteroid supplements, e.g., prednisone or prednisolone; agents against goiter formation, e.g., colchicine or allopurinol; antiepileptics, e.g., phenyloine sodium; agents against dehydration, e.g., electrolyte supplements; antiseptics, e.g., cetylpyridinium chloride; NSAIDs, e.g., acetaminophen, ibuprofen, naproxen, or salts thereof; gastrointestinal active agents, e.g., loperamide and famotidine; various alkaloids, e.g., codeine phosphate, codeine sulfate, or morphine; supplements for trace elements, e.g., sodium chloride, zinc chloride, calcium carbonate, magnesium oxide, and other alkali metal salts and alkali earth metal salts; vitamins; ion-exchange resins, e.g., cholestyramine; cholesterol-depressant and lipid-lowering substances; antiarrhythmics, e.g., N-acetylprocainamide; and expectorants, e.g., guaifenesin.

Active substances which have a particularly unpleasant taste include antibacterial agents such as ciprofloxacin, ofloxacin, and pefloxacin; antiepileptics such as zonisamide; macrolide antibiotics such as erythromycin; beta-lactam antibiotics such as penicillins and cephalosporins; psychotropic active substances such as chlorpromazine; active substances such as sulpyrine; and agents active against ulcers, such as cimetidine. In another embodiment, the pharmaceutical composition of the present invention comprises at least one amino acid selected from the group consisting of glycine, L-alanine, L-arginine, L-aspartic acid, L-cystine, L-glutamic acid, L-glutamine, L-histidine, L-isoleucine, L-leucine, L-lysine, L-methionine, L-ornithine, L-phenylalanine, L-proline, L-serine, L-threonine, L-tryptophan, L-tyrosine, L-valine, creatine, and mixtures thereof.

The pharmaceutical compositions of the present invention are administered to a subject in any form suitable to achieve their intended purpose. Preferably, however, the composition is one which can be administered buccally or orally. Alternatively, the pharmaceutical composition can be an oral or nasal spray. The subject is any animal, such as a human, although the invention is not intended to be so limited. Other suitable animals include canines, felines, dogs, cats, livestock, horses, cattle, sheep, and the like. A veterinary composition, as used herein, refers to a pharmaceutical composition that suitable for non-human animals. Such veterinary compositions are known in the art.

In another embodiment, the pharmaceutical composition is a liquid dosage form for oral administration, including pharmaceutically acceptable emulsions, solutions, suspensions, syrups, and elixirs. In addition to the active compounds, the liquid dosage forms can contain inert diluents commonly used in the art such as, for example, water or other solvents, solubilizing agents and emulsifiers such as ethyl alcohol, isopropyl alcohol, ethyl carbonate, ethyl acetate, benzyl alcohol, benzyl benzoate, propylene glycol, 1,3-butylene glycol, dimethyl formamide, oils (in particular, cottonseed, groundnut, corn, germ, olive, castor, and sesame oils), glycerol, tetrahydrofurfuryl alcohol, polyethylene glycols and fatty acid esters of sorbitan, and mixtures thereof. Suspensions, in addition to the active compounds, can contain suspending agents as, for example, ethoxylated isostearyl alcohols, polyoxyethylene sorbitol and sorbitan esters, microcrystalline cellulose, aluminum metahydroxide, bentonite, agar-agar, and tragacanth, and mixtures thereof.

The pharmaceutical composition of the present invention can be in the form of a chewable tablet. Chewable tablets are known in the art. See, e.g., U.S. Pat. Nos. 4,684,534 and 6,060,078, each of which is incorporated by reference in its entirety. Any kind of medicament can be contained in the chewable tablet, preferably a medicament of bitter taste, natural plant extracts or other organic compounds. More preferably, vitamins such as vitamin A, vitamin B, vitamin B₁, vitamin B₂, vitamin B₆, vitamin C, vitamin E and vitamin K; natural plant extracts such as Sohgunjung-tang extracts, Sipchundaebo-tang extracts and Eleutherococcus senticosus extracts; organic compounds such as dimenhydrinate, meclazine, acetaminophen, aspirin, phenylpropanolamine, and cetylpyridinium chloride; or gastrointestinal agents such as dried aluminum hydroxide gel, domperidone, soluble azulene, L-glutamine and hydrotalcite can be contained in the core.

The pharmaceutical composition of the present invention can be an orally disintegrating composition. Orally disintegrating tablets are known in the art. See, e.g., U.S. Pat. Nos. 6,368,625 and 6,316,029, each of which is hereby incorporated by reference in its entirety.

The pharmaceutical composition of the present invention can be a nasal composition, comprising a carbohydrate sweetener and one or more Reb C polymorphs. Nasal sprays are known in the art. See, e.g., U.S. Pat. No. 6,187,332. Addition of one or more Reb C polymorphs to a nasal spray can reduce the experience of an unpleasant taste associated with the composition of the nasal spray.

The pharmaceutical composition of the present invention can be a solid dosage form, comprising a carbohydrate sweetener and one or more Reb C polymorphs and a water and/or saliva activated effervescent granule, such as one having a controllable rate of effervescence. The effervescent composition can further comprise a pharmaceutically active compound. Effervescent pharmaceutical compositions are known in the art. See, e.g., U.S. Pat. No. 6,649,186, which is incorporated by reference in its entirety. The effervescent composition can be used in pharmaceutical, veterinary, horticultural, household, food, culinary, pesticidal, agricultural, cosmetic, herbicidal, industrial, cleansing, confectionery and flavoring applications. Formulations incorporating the effervescent composition comprising one or more Reb C polymorphs can further include one or more additional adjuvants and/or active ingredients which can be chosen from those known in the art, including flavors, diluents, colors, binders, filler, surfactant, disintegrant, stabilizer, compaction vehicles, and non-effervescent disintegrants.

The pharmaceutical composition can be a film-shaped or wafer-shaped pharmaceutical composition. Such a film-shaped or wafer-shaped pharmaceutical composition can be configured, for example, as quickly disintegrating administration forms, e.g., administration forms disintegrating within a period of 1 second up to 3 minutes, or as slowly disintegrating administration forms, e.g., administration forms disintegrating within a period of 3 to 15 minutes. The indicated disintegration times can be set to the above-mentioned ranges by using, for example, matrix-forming polymers which have different disintegrating, or solubility, characteristics. Thus, by mixing the corresponding polymer components, the disintegration time can be adjusted. In addition, disintegrants are known which “draw” water into the matrix and cause the matrix to burst open from within. As a consequence, certain embodiments of the invention include such disintegrants for the purpose of adjusting the disintegration time.

Suitable are polymers for use in the film-shaped or wafer-shaped pharmaceutical composition include cellulose derivatives, polyvinyl alcohol (e.g. MOWIOL™), polyacrylates, polyvinyl pyrrolidone, cellulose ethers, such as ethyl cellulose, as well as polyvinyl alcohol, polyurethane, polymethacrylates, polymethyl methacrylates and derivatives and copolymerisates of the aforementioned polymers.

In certain embodiments, the total thickness of the film-shaped or wafer-shaped pharmaceutical composition according to the invention is preferably 5 μm up to 10 mm, preferably 30 μm to 2 mm, and with particular preference 0.1 mm to 1 mm. The pharmaceutical preparations can be round, oval, elliptic, triangular, quadrangular or polygonal shape, but they can also have any rounded shape.

In one embodiment, the pharmaceutical composition can be a gum base formulation comprising a medicament or agent contained, a carbohydrate sweetener and one or more Reb C polymorphs in a coating that surrounds the gum base formulation. Preferably, the coating comprises at least 50% by weight of the entire product. As the center is chewed, the medicament or agent is released into the saliva. For example, U.S. Pat. No. 6,773,716, which is incorporated herein by reference in its entirety, discloses a suitable medicament or agent contained in a coating that surrounds a gum base formulation. It has been found that with respect to certain medicaments or agents that can have an astringent or bitter taste that by adding a sweet taste enhancing agent to the formulation, that a much more palatable formulation, including the medicament, can be provided. In this regard, even though the medicament in, for example, its powder form may be bitter or have an offensive taste, the matrix used as the coating of the present invention, including the enhancing agent, will afford a product having acceptable medicinal properties.

The pharmaceutical composition of the present invention can be in the form of an aerosol. The aerosol composition can further comprise pharmaceutically active agent. Aerosol compositions are known in the art. See, e.g., U.S. Pat. No. 5,011,678, which is hereby incorporated by reference in its entirety. As a nonlimiting example, an aerosol composition according to the present invention can comprise a medically effective amount of a pharmaceutically active substance, one or more carbohydrate sweeteners, one or more Reb C polymorphs and a biocompatible propellant, such as a (hydro/fluoro)carbon propellant.

In one embodiment of the present invention, the pharmaceutical composition is a nutritional composition. Examples of nutritional compositions having an undesirable taste include, but are not necessarily limited to, enteral nutrition products for treatment of nutritional deficit, trauma, surgery, Crohn's disease, renal disease, hypertension, obesity and the like, to promote athletic performance, muscle enhancement or general well being or inborn errors of metabolism such as phenylketonuria. In particular, such nutritional formulations can contain one or more amino acids which have a bitter or metallic taste or aftertaste. Such amino acids include, but are not limited to, an essential amino acids selected from the group consisting of L isomers of leucine, isoleucine, histidine, lysine, methionine, phenylalanine, threonine, tryptophan, tyrosine, and valine.

In one embodiment, the sweet taste of the pharmaceutical composition or nutritional composition of the present invention is being enhanced by one or more Reb C polymorphs by at least about 10%, 20%, 30%, 40%, 50%, 60%, 70%, 80%, 90%, or 95%, or from about 60% to about 99%, or alternatively from about 20% to about 50%.

In one embodiment, the consumable of the present invention is a dental hygienic composition, comprising a carbohydrate sweetener and one or more Reb C polymorphs in an amount sufficient to enhance the sweet taste of the carbohydrate sweetener without exhibiting any off-taste. Dental hygienic compositions are known in the art and include, but are not necessarily limited to, toothpaste, mouthwash, plaque rinse, dental floss, dental pain relievers (such as ANBESOL™), and the like. In one embodiment, the dental hygienic composition comprises one carbohydrate sweetener. In another embodiment, the dental hygienic composition comprises more than one carbohydrate sweetener. In certain embodiments, the dental hygienic composition comprises sucrose and corn syrup, or it comprises sucrose and aspartame.

In another embodiment, the consumable of the present invention is a cosmetic product comprising a carbohydrate sweetener and one or more Reb C polymorphs. For example, but not by way of limitation, the cosmetic product can be a face cream, lipstick, lip gloss, and the like. Other suitable compositions of the invention include lip balm, such as CHAPSTICK® or BURT'S BEESWAX® Lip Balm, further comprising one or more Reb C polymorphs.

The present invention is also directed to various, useful consumables comprising one or more Reb C polymorphs described above.

In one embodiment, the present invention is directed to a food product comprising a carbohydrate sweetener and one or more Reb C polymorphs. Preferably, the food product is one which exhibits a sweet taste (i.e., inherently contains a carbohydrate sweetener) and/or to which a carbohydrate sweetener has been added. The food product comprises one or more Reb C polymorphs in an amount sufficient to enhance the sweet taste without exhibiting an off-taste. Specific carbohydrate sweeteners have been described above. Specific food products in which an enhanced sweet taste is desired include, but are not limited to, cakes, cookies, confectionaries, such as candies, gums and chocolates, creams, icing, ice cream, pies and breads. Specific food products which are beverages include soft drinks, juices and other fruit drinks, sports drinks such as GATORADE®, coffee, teas, iced teas, cola, alcoholic beverages and KOOL-AID®.

In certain aspects, the present invention provides methods and compositions for enabling one to prepare consumable products, such as food and pharmaceutical products, which retain a desired sweetness but contain lower amounts of a carbohydrate sweetener, such as sugar, and in some cases fewer calories.

In one aspect, the food product of the present invention comprises a tabletop sweetener composition, comprising (i) at least one carbohydrate sweetener, (ii) one or more rebaudioside C polymorphs, especially rebaudioside C crystalline Form I, and (iii) optionally a bulking agent, wherein the one or more rebaudioside C polymorphs are each present in an amount effective to synergistically enhance the sweetness of the carbohydrate sweetener.

In one embodiment, one serving size of the tabletop sweetener of the present invention provides a sweetness intensity equivalent to a 5-12% (w/v-%) sucrose solution. In one embodiment, one serving size of the tabletop sweetener of the present invention provides a sweetness intensity equivalent to an 8-12% (w/v-%) sucrose solution.

In one embodiment, the tabletop sweetener of the present invention does not comprise a bulking agent. In one embodiment, the tabletop sweetener of the present invention comprises a bulking agent. Suitable bulking agents include maltodextrin, polydextrose, fructooligosaccharides, cellulose and cellulose derivatives, isomalt, maltose, tagatose, lactose, inulin, glycerol, propylene glycol, polyols, xylose, ribulose, mannose, and the like. The amount of bulking agent used is typically the smallest amount that provides for accurate delivery. Especially suitable bulking agents include dextrose and maltodextrin.

In one embodiment, the tabletop sweetener composition of the present invention comprises an anti-caking agent or a flow agent. As used herein, the phrase “anti-caking agent” and “flow agent” refer to any composition which prevents, reduces, inhibits, or suppresses at least one sweetener molecule from attaching, binding or contacting to another sweetener molecule. Alternatively, anti-caking agent may refer to any composition which assists in content uniformity and uniform dissolution. Suitable anti-caking agents include cream of tartar, calcium cilicate, silicon dioxide, microcrystalline cellulose (Avicel®), and tricalcium phosphate. In one embodiment, the anti-caking agents are present in the tabletop sweetener composition in an amount from about 0.001% to about 3% by weight of the tabletop sweetener composition.

In one embodiment, the tabletop sweetener composition of the present invention comprises a flavor or aroma. As used herein, the term “flavor” means any food-grade material that may be added to the compositions of the present invention to provide a desired flavor to a foodstuff. Suitable flavors include, for example, cream, hazelnut, vanilla, chocolate, cinnamon, pecan, lemon, lime, raspberry, peach, mango, vanillin, butter, butterscotch, tea, orange, tangerine, caramel, strawberry, watermelon, bubblegum, cantaloupe, guava, kiwi, papaya, coconut, mint, spearmint, and combinations thereof.

As used herein, the term “aroma” means any food-grade volatile substance that may be employed to produce a desired scent, for example, when mixed with a foodstuff. Suitable aromas include, for example, essential oils (citrus oil), expressed oils (orange oil), distilled oils (rose oil), extracts (fruits), anethole (liquorice, anise seed, ouzo, fennel), anisole (anise seed), benzaldehyde (marzipan, almond), benzyl alcohol (marzipan, almond), camphor (cinnamomum camphora), cinnamaldehyde (cinnamon), citral (citronella oil, lemon oil), d-limonene (orange), ethyl butanoate (pineapple), eugenol (clove oil), furaneol (strawberry), furfural (caramel), linalool (coriander, rose wood), menthol (peppermint), methyl butanoate (apple, pineapple), methyl salicylate (oil of wintergreen), neral (orange flowers), nerolin (orange flowers), pentyl butanoate (pear, apricot), pentyl pentanoate (apple, pineapple), sotolon (maple syrup, curry, fennugreek), strawberry ketone (strawberry), substituted pyrazines, e.g., 2-ethoxy-3-isopropylpyrazine; 2-methoxy-3-sec-butylpyrazine; and 2-methoxy-3-methylpyrazine (toasted seeds of fenugreek, cumin, and coriander), thujone (juniper, common sage, Nootka cypress, and wormwood), thymol (camphor-like), trimethylamine (fish), vanillin (vanilla), and combinations thereof. Preferred aroma components according to the present invention include, essential oils (citrus oil), expressed oils (orange oil), distilled oils (rose oil), extracts (fruits), benzaldehyde, d-limonene, furfural, menthol, methyl butanoate, pentyl butanoate, salts, and combinations thereof. The aroma may be present in any amount in the composition. Preferably, the aroma component is present in an amount from about 2- to about 10-times the detectable amount. More preferably, the aroma component is present in an amount from about 2-to about 5-times the detectable amount. As used herein, unless otherwise indicated, the term “detectable amount” is the amount of the aroma component required to produce a scent detectable in the foodstuff.

In one embodiment, the tabletop sweetener composition of the present invention comprises a binder. As used herein, the term “binder” refers to any food-grade material that is suitable for facilitating the pressing and formation of tablets. Suitable binders include any conventional binders as long as the binder does not substantially interfere with the self-mixing or the organoleptic properties of the foodstuff, such as, for example, microcrystalline cellulose, gum traganth, gelatin, leucine, lactose, and combinations thereof. The binder may be present in an amount of from about 10% to about 15% by weight of the total composition.

Tabletop sweetener compositions of the present invention can be packaged in numerous different forms, such as, for example, powder form, granular form, sachets, packets, tablets, pellets, cubes, solids, liquids, dissolvable sweetening strips, and sprays.

In one embodiment, a tabletop sweetener comprises a single serving (portion control) packet comprising a dry-blend of a sweetener composition formulation. Dry-blend formulations generally comprise powder or granules. The tabletop sweetener packet may be of any size, for example about 2.5 by 1.5 inches and hold approximately 1 gram of a sweetener composition of the present invention having a sweetness equivalent to 2 teaspoons of granulated sugar (about 8 g). In one embodiment, a dry-blend tabletop sweetener formulation comprises one or more Reb C polymorphs, each independently in an amount of from about 1% (w/w-%) to about 10% (w/w-%) of the tabletop sweetener composition.

Solid tabletop sweetener forms include cubes and tablets. For example, conventional cubes are equivalent in size of a standard cube of granulated sugar, which is approximately 2.2×2.2×2.2 cm³ and weigh approximately 8 grams. In one embodiment, a solid tabletop sweetener is in the form of a tablet or any other form known to those skilled in the art.

In one embodiment, the tabletop sweetener composition of the present invention is in the form of a liquid. In this aspect of the invention, one or more Reb C polymorphs, and at least one carbohydrate sweetener are combined with a liquid carrier. Suitable non-limiting examples of carriers for liquid tabletop sweeteners include water, alcohol, polyol, glycerin base or citric acid base dissolved in water, and mixtures thereof.

The sweetness equivalent of a tabletop sweetener composition for any of the forms described herein or known in the art can be varied to obtain a desired sweetness profile. For example, a tabletop sweetener composition can comprise a sweetness comparable to that of an equivalent amount of standard sugar. In another embodiment, the tabletop sweetener composition can comprise a sweetness up to 100 times that of an equivalent amount of sugar. In another embodiment, the tabletop sweetener composition can comprise a sweetness of up to 90 times, 80 times, 70 times, 60 times, 50 times, 40 times, 30 times, 20 times, 9 times, 8 times, 7 times, 6 times, 5 times, 4 times, 3 times, and 2 times that of an equivalent amount of sugar.

In one embodiment, the tabletop sweetener composition can also be formulated for targeted uses such as, for example, in beverage, food, pharmaceutical, nutraceutical, cosmetics, and in any other products that may be sweetened. For example, a tabletop sweetener composition for baking can be formulated having additional protecting agents, such as encapsulants. Other forms will be readily apparent to those skilled in the tabletop sweetener art.

Commonly used methods for making powder or granulated sweetener formulations for packets include fluid bed agglomeration process. Other methods for making tabletop sweetener compositions are well known to those of ordinary skill in the art.

In one aspect, the present invention provides a method of making a tabletop sweetener composition, comprising including (i) at least one carbohydrate sweetener, (ii) one or more Reb C polymorphs, and (iii) optionally a bulking agent. In one embodiment, the one or more rebaudioside C polymorphs are included in an amount effective to synergistically enhance the sweetness of the carbohydrate sweetener.

Unless otherwise specified, percentages (% s) are by weight.

The following examples are illustrative, but not limiting, of the compounds, compositions, and methods of the present invention. Suitable modifications and adaptations of the variety of conditions and parameters normally encountered in clinical therapy and which are obvious to those skilled in the art in view of this disclosure are within the spirit and scope of the invention.

### Example 1

Three samples of Reb C (93.3% HPLC purity; ChromaDex®) were placed in separate wells of a polypropylene 96-well V-bottom plate as follows: well A1, 5.8 mg; well D1, 5.5 mg; well G1, 5.4 mg. Absolute ethanol was added to A1 (75% of ˜300 μL well volume), anhydrous methanol was added to D1 (75% well volume), and isopropanol was added to G1 (50% well volume) followed by anhydrous methanol (25% well volume). The plate was gently agitated until the material in the three wells had fully dissolved. The plate was left uncovered and the solvents were allowed to evaporate overnight at room temperature. This produced glass-like, non-crystalline, material in all three wells. The material in each well was once again dissolved using the same process as described above. The 96-well V-bottom plate containing the dissolved Reb C samples was covered by a second empty plate to slow the evaporation rate and this was left at room temperature overnight. Colorless/white crystalline solid was obtained in each well. This material was transferred into vials marked as A1, D1 and G1, corresponding to the well locations. To a fourth vial labeled B2 was added 5 mg of the starting Reb C sample obtained from ChromaDex®. The four vials were sent to XRD US (Cold Spring, N.Y.) for powder x-ray diffraction analysis.

Samples of Reb C crystals taken from the A1, D1, and G1 vials were combined and dissolved in a minimal amount of methanol to produce a Reb C/methanol solution. The Reb C/methanol solution was then diluted with isopropanol at a volume ratio of 1:9. The methanol/isopropanol recrystallized Reb C sample, the original Reb C sample obtained from ChromaDex® and the Reb C samples forwarded in vials A1, D1, and G1 were analyzed by powder diffraction using a Shimadzu XRD-6000 X-ray powder diffractometer using Cu Kα (1.54 Å) radiation. The instrument was equipped with a long fine focus X-ray tube. The tube voltage and amperage were set to 40 kV and 40 mA, respectively. The divergence and scattering slits were set at 1°, and the receiving slit was set at 0.3°. Diffracted radiation was detected by a Shimadzu SC-1001 scintillation detector. The samples were analyzed by methods known to those of skill in the art using the TREOR software package. Reb C crystalline Form I corresponds to the methanol/isopropanol recrystallized Reb C sample and comprises significant peaks at d-spacing values: 8.6, 9.8, 12.6, 13.6, 13.9, 14.2, 14.9, 15.6, 17.0, 17.4, 18.2, 19.9, 21.3, 22.6, 23.3, 25.5, 27.2, 28.4, 28.9, and 30.0. FIG. 2 depicts all of the significant XRPD peaks associated with Reb C crystalline Form I. FIG. 1 compares the XRPD spectra associated with Reb C crystalline Form I (methanol/isopropanol recrystallized Reb C sample), the original Reb C sample obtained from ChromaDex® and the Reb C crystal samples forwarded in vials A1, D1, and G1.

### Example 2

A Crude Stevia extract solid or a crude Reb C solid (5 g), ethanol (95%, 12.5 mL), methanol (6 mL) and water (2 mL) can be combined and heated to reflux for 10 minutes. The clear solution can be cooled to 22° C. The solution can be seeded with 10 mg of 93-98% pure Reb C crystalline Form I crystals and the mixture can be left at 22° C. for 16 hours. Resulting white crystalline product can be filtered, washed twice with ethanol-methanol (5 mL, 4:1, v/v) mixture and dried in a vacuum oven at 50° C. for 16-24 hours under reduced pressure (20 mm) to yield purified Reb C product. The purity of the resulting Reb C product can be assessed by HPLC.

### Example 3

A Crude Stevia extract solid or a crude Reb C solid (5 g), propanol (95%, 12.5 mL) and methanol (7.5 mL) can be combined and heated to reflux for 10 minutes. The clear solution can be cooled to 22° C. The solution can be seeded with 10 mg of 93-98% pure Reb C crystalline Form I crystals and the mixture can be left at 22° C. for 16 hours. Resulting white crystalline product can be filtered, washed twice with ethanol-methanol (5 mL, 4:1, v/v) mixture and dried in a vacuum oven at 50° C. for 16-24 hours under reduced pressure (20 mm) to yield purified Reb C product. The purity of the resulting Reb C product can be assessed by HPLC.

Having now fully described this invention, it will be understood by those of ordinary skill in the art that the same can be performed within a wide and equivalent range of conditions, formulations and other parameters without affecting the scope of the invention or any embodiment thereof. All patents, published patent applications, and publications cited herein are fully incorporated by reference herein in their entirety.

What is claimed is:

1. A method for producing isolated rebaudioside C crystalline Form I comprising:
(a) adding a crystallization solution comprising methanol and isopropanol to a substantially pure rebaudioside C solid to completely dissolve the rebaudioside; and
(b) recovering isolated rebaudioside C crystalline Form I crystals.

2. The method of claim 1, wherein the methanol and isopropanol are present in a weight ratio ranging from about 5 parts to about 1 part isopropanol to about 1 part methanol.

3. Isolated rebaudioside C crystalline Form I produced by the method of claim 1, wherein the rebaudioside C crystalline Form I, which has a powder X-ray diffraction pattern at Cu Kα wavelength 1.54 Å as shown in FIG. 2.

4. The isolated rebaudioside C crystalline Form I of claim 3, wherein the d-spacing distances (Å) of significant peaks as determined by powder X-ray diffraction are: 8.6, 9.8, 12.6, 13.6, 13.9, 14.2, 14.9, 15.6, 17.0, 17.4, 18.2, 19.9, 21.3, 22.6, 23.3, 25.5, 27.2, 28.4, 28.9, and 30.0.

5. A method of enhancing a sweet taste of a carbohydrate sweetener, comprising administering to a subject the carbohydrate sweetener and isolated rebaudioside C crystalline Form I of claim 3 in an amount effective to provide the sweet taste enhancing effect without exhibiting an off-taste.

6. The method of claim 5, wherein the carbohydrate sweetener and isolated rebaudioside C crystalline Form I are administered in a consumable.

7. The method of claim 5, wherein the consumable is a food product, pharmaceutical composition, a dietary supplement, a nutraceutical, a dental hygienic composition, a tabletop sweetener, or a cosmetic product.

8. The method of claim 5, wherein isolated rebaudioside C crystalline Form I is present at a concentration of from about 150 μM to about 600 μM.

9. A consumable comprising a carbohydrate sweetener and isolated rebaudioside C crystalline Form I of claim 3 in an amount effective to enhance the sweet taste of the carbohydrate sweetener without exhibiting an off-taste.

10. The consumable of claim 9, wherein the isolated rebaudioside C crystalline Form I is present at a concentration of from about 150 μM to about 600 μM.

11. The consumable of claim 9, wherein the consumable is a food product, pharmaceutical composition, a dietary supplement, a nutraceutical, a dental hygienic composition, a tabletop sweetener or a cosmetic product.

12. A method of decreasing the amount of a carbohydrate sweetener in a consumable, comprising adding the isolated rebaudioside C crystalline Form I of claim 3 to the consumable and reducing the amount of the carbohydrate sweetener needed to exhibit a given level of sweetness.

13. The method of claim 12, wherein the consumable is a food product, pharmaceutical composition, a dietary supplement, a nutraceutical, a dental hygienic composition, a tabletop sweetener or a cosmetic product.

14. The method of claim 12, wherein the isolated rebaudioside C crystalline Form I is present at a concentration of from about 150 μM to about 600 μM.

15. A method of enhancing the sweetness of a consumable comprising a carbohydrate sweetener, comprising adding isolated rebaudioside C crystalline Form I of claim 3 to the consumable in an amount effective to enhance the sweetness of the consumable.

16. The method of claim 15, wherein the isolated rebaudioside C crystalline Form I is added to the consumable in an amount to obtain a concentration of from about 150 μM to about 600 μM.

17. The method of claim 15, wherein about 0.1 to 0.5 g of isolated rebaudioside C crystalline Form I is added for every 50 to 100 g of the carbohydrate sweetener.

18. The method of claim 15, wherein the consumable is a food product, pharmaceutical composition, a dietary supplement, a nutraceutical, a dental hygienic composition, a tabletop sweetener or a cosmetic product.
